(19) United States
(12) Patent Application Publication
(10) Pub. No.: US 2019/0265232 A1
(43) Pub. Date: Aug. 29, 2019

(54) METHODS AND KITS FOR ANALYSING THE STEADY-STATE ACTIVATION OR INHIBITION OF ITAM SIGNALLING OF IMMUNORECEPTORS IN BLOOD LEUKOCYTES

(71) Applicants: INSERM (INSTITUT NATIONAL DE LA SANTE ET DE LA RECHERCHE MEDICALE), Paris (FR); UNIVERSITÉ PARIS DIDEROT - PARIS 7, Paris (FR); ASSISTANCE PUBLIQUE-HÔPITAUX DE PARIS (APHP); CENTRE NATIONAL DE LA RECHERCHE SCIENTIFIQUE (CNRS), Paris (FR)

(72) Inventors: Renato MONTEIRO, Paris Cedex 18 (FR); Sanae BEN MKADDEM, Paris Cedex 18 (FR); Eric DAUGAS, Paris Cedex 18 (FR)

(21) Appl. No.: 16/308,299

(22) PCT Filed: Jun. 9, 2017
(86) PCT No.: PCT/EP2017/064095

§ 371 Date: Dec. 7, 2018

(30) Foreign Application Priority Data
Jun. 10, 2016 (EP) 16305683.1

(51) Int. Cl. G01N 33/50

(52) U.S. Cl. CPC G01N 33/5047; G01N 2800/24; G01N 2800/52; G01N 33/5041

(57) ABSTRACT

The present invention relates to methods and kits for analysing the steady-state activation or inhibition of ITAM signalling of immunoreceptors in blood leukocytes. The inventors investigated that low valency ligands induced ITAMi signals by FcR, but also BCR and TCR, that were driven by Lyn or Lck recruitment followed by SHP-1 activation resulting in inhibition of heterologous activating receptors. In contrast, Fyn was required for multivalent ligand induced ITAM signals driving Syk or Zap-70 recruitment resulting in cell activating functions. In particular, the present invention relates to a method for analysing the activating or inhibiting steady-state of ITAM signalling of immunoreceptors in a population of leukocytes comprising by determining whether the immunoreceptors are associated with a Fyn-kinase activity or are associated with a Lyn/Lck kinase activity.

### FIELD OF THE INVENTION

The present invention relates to methods and kits for analysing the steady-state activation or inhibition of ITAM signalling of immunoreceptors in blood leukocytes.

### BACKGROUND OF THE INVENTION

Autoimmune and inflammatory diseases present as a major public health problem worldwide. In particular, autoimmune and inflammatory renal diseases, mostly glomerular diseases, are the leading cause of renal transplantation in France and the third leading cause of renal replacement therapy by dialysis after hypertension and diabetes (REIN, Rapport Annuel 2010 (Rein rapports 2011, 2012, www.agence-biomedecine.fr)). The most prevalent glomerulonephritis (GN) are linked to dysfunctional immune system such as well-characterized IgA nephropathy (IgA-N or Berger's disease), lupus nephritis (LN) and renal-associated vasculitis such as Anti-Neutrophil Cytoplasmic Antibodies (ANCA) associated vasculitis (AAV). Patients present with a relative silent evolution and late appearance of symptoms, independent of the type of immune involvement. To date, diagnosis is only possible through invasive investigations, mainly renal histology comprising evaluation of the presence of immunoglobulins and complement deposits in the glomeruli. There is thus an unmet medical need of non-invasive methods suitable for diagnosing inflammatory auto-immune diseases.

The immune system is controlled by a finely tuned network of regulatory mechanisms to maintain homeostasis (Bezbradica and Medzhitov, 2012). One axis of regulation comprises immunoreceptor tyrosine-based activation motif (ITAM)-containing immunoreceptors such as the T- and B-cell antigen receptors (TCR, BCR) and Fc receptors (FcR) as well as an expanding family of other ITAM-associated receptors with various functions in immunity (Abram and Lowell, 2007; Hamerman and Lanier, 2006). The ITAM motif is defined by two consecutive Yxx[L/I sequences separated by 6 to 12 amino acids. It is found in the cytoplasmic domains of several transmembrane adapter molecules, such as the common γ subunit of FcR (FcRγ), the Igα and Igβ subunits of the BCR, and the γ, δ, ε and ζ subunits of the TCR-associated CD3 complex (Bezbradica and Medzhitov, 2012; Reth, 1989) as well as in the cytoplasmic tail of other receptors such as the FcγRIIA (Hogarth, 2002). Cellular responses after Fc-receptor triggering depend on ligand valency. High valency ligand interaction mediated receptor clustering induces phosphorylation on ITAM-tyrosine residues by membrane-localized and receptor-associated Src-family kinases (SFK). Phosphorylated ITAMs serve as a docking site for recruitment of Syk or Zap70 kinases launching the inflammatory responses to fight the insult and restore homeostasis, but in case of ill-regulation or chronic stimulation can also result in autoimmune and inflammatory diseases (Bezbradica and Medzhitov, 2012; Getahun and Cambier, 2015). In addition ITAM-bearing receptors has been described to be inhibitory following low valency interactions, which induces anergy (BCR, TCR) as well as inhibitory signaling towards autologous/heterologous receptors in the case of FcR. For the latter this mechanism has been named inhibitory ITAM (ITAMi) (Blank et al., 2009; Pasquier et al., 2005). Different FcR, such as FcαRI, FcγRIIA and FcγRIIIA were shown to act as bi-functional receptors triggering inhibitory signals towards a whole array of activating receptors, a property that can be exploited to reduce the susceptibility to autoimmune and inflammatory diseases (Aloulou et al., 2012; Ben Mkaddem et al., 2014; Kanamaru et al., 2008; Pasquier et al., 2005; Pinheiro da Silva et al., 2007).

Induction of ITAMi signals of FcR by weakly binding ligand (low affinity, low avidity, low valency) depends on the recruitment of inhibitory SHP-1 phosphatase (Blank et al., 2009; Mkaddem et al., 2014). BCR and TCR have also been reported to recruit SHP-1 phosphatase upon interaction with low valency ligands (Getahun et al., 2016; Stefanova et al., 2003). For the BCR, high phosphatase activity (SHP-1 and SHIP-1) has been shown to short-circuit signaling during the selection process of relevant ligands in germinal centers (Khalil et al., 2012). In T cells, specific deletion of SHP-1 in CD4 T-cells via a floxed Shp1^{fl/fl} CD4-cre system in mice demonstrated a key role for SHP-1 in negatively regulating the responsiveness of CD4 T-cells to interleukin-4 signaling, and hence maintenance of a TH1 phenotype (Johnson et al., 2013). In other haematopoietic lineages including neutrophils and dendritic cells, deletion of SHP-1 was associated with a variety of pathologies (Abram et al., 2013; Croker et al., 2008; Pao et al., 2007). Together, these evidences support an important role of ITAMi mediated SHP-1 recruitment in the maintenance of immune homeostasis.

ITAM-bearing receptors are associated with SFKs such as Lyn, Lck, Fyn. They are the relevant kinases responsible for ITAM phosphorylation upon receptor aggregation leading to Syk/Zap-70 recruitment and further signal propagation via downstream effectors such as LAT, PI3-kinase and phospholipase C-γ etc (Iwashima et al., 1994; Packard and Cambier, 2013). However, their precise role and functional coordination of each SFK in ITAM signalling still remains obscure. Both redundant and independent SFK functions have been described (Palacios and Weiss, 2004). In mast cell FIERI, it has been demonstrated that Lyn is a negative regulator of anaphylaxis (Odom et al., 2004), while Fyn was shown to be a positive regulator (Parravicini et al., 2002). Likewise in T cells, initial studies reported hyporesponsiveness of Fyn^{-/-} cells upon anti-CD3 stimulation (Appleby et al., 1992), which do not activate Lck to the extent occurring in mature peripheral T cells stimulated with anti-CD3 plus CD4/CD8 or antigen (Holdorf et al., 1999; Luo and Sefton, 1990). Indeed, Fyn^{-/-} cells stimulated with antigen or by anti-CD3/CD4 showed no defects in activation (Sugie et al., 2004), with the exception of responses of transgenic Fyn^{-/-} cells to low-affinity ligands (Utting et al., 1998). Concerning the BCR signaling, Lyn activation was shown to induce distinct outcomes depending on the strength of BCR signal, the developmental stage of the B cell and coreceptor function. Indeed Lyn was shown to play both positive and negative roles in BCR-mediated signaling (Gauld and Cambier, 2004). Aged Lyn-deficient mice display high levels of serum immunoglobulins (including autoantibodies), their B cells are hyperresponsive to IL-4 and CD40 engagement (Hibbs et al., 1995; Janas et al., 1999; Nishizumi et al., 1995) and significant increase in basophil numbers in the lymph nodes, blood and spleen (Charles et al., 2009). Together, these studies suggest that the precise role and functional coordination of Lyn/Lck and Fyn in the control of different ITAM signaling pathways mediated by the engagement of FcRs, BCR or TCR by either high or low valency ligands still remains obscure.

### SUMMARY OF THE INVENTION

The present invention relates to methods and kits for analysing the steady-state activation or inhibition of ITAM signalling of immunoreceptors in blood leukocytes. In particular, the present invention is defined by the claims.

### DETAILED DESCRIPTION OF THE INVENTION

Here, the inventors investigated that low valency ligands induced ITAMi signals by FcR, but also BCR and TCR, that were driven by Lyn or Lck recruitment followed by SHP-1 activation resulting in inhibition of heterologous activating receptors. In contrast, Fyn was required for multivalent ligand induced ITAM signals driving Syk or Zap-70 recruitment resulting in cell activating functions. Mechanistically, Fyn inhibited ITAMi signaling via SHP-1 serine phosphorylation through the PI3K-PKCα pathway. FcγRIIA^{Tg}/Lyn^{-/-} mice developed lethal autoimmune nephritis and severe arthritis with massive tissue infiltration of hyperactivated leukocytes, whereas Fyn-deficient mice were protected. Patients with lupus nephritis, but not healthy subjects, exhibited a typical FcγRIIA-associated ITAM signature with strong recruitment of Fyn and weak recruitment of Lyn associated with the activation of PKCα and phosphorylation of SHP-1 on serine 591. Inversely, healthy subjects display FcγRIIA-associated ITAMi signature with strong recruitment of Lyn but not Fyn associated with the phosphorylation of SHP-1 on tyrosine 536. Therefore, Fyn acts as an active switch inducing inflammation turning off Lyn (Lck)-dependent ITAMi signals that control immune homeostasis. These findings demonstrate the distinct roles of antigen receptor-associated Src family kinases in regulating homeostatic and inflammatory conditions.

Accordingly, the first object of the present invention relates to a method for analysing the activating or inhibiting steady-state of ITAM signalling (ITAMa or ITAMi) of immunoreceptors in a population of leukocytes comprising i) determining whether the immunoreceptors are associated with a Fyn-kinase activity or are associated with a Lyn/Lck kinase activity and ii) concluding that the immunoreceptors are in a steady state activation of ITAM signalling (ITAMa) when they are associated with a Fyn kinase activity or concluding that the immunoreceptors are in a steady state inhibition of ITAM signalling (ITAMi) when they are associated with a Lyn/Lck kinase activity.

As used herein the term “leukocyte” has its general meaning in the art and refers to any type of white blood cell. Leukocytes may be peripheral leukocytes. Examples of leukocytes include, for example granulocytes (e.g., neutrophils, eosinophils, basophils), mononuclear phagocytes, and lymphocytes (e.g., B cells, T cells, natural killer (NK) cells). Leukocytes may be isolated in accordance with any suitable technique. Typically, these cells can be extracted from whole blood using Ficoll, a hydrophilic polysaccharide that separates layers of blood, with the leukocytes forming a cell ring under a layer of plasma. Additionally, leukocytes can be extracted from whole blood using a hypotonic lysis which will preferentially lyse red blood cells. Such procedures are known to the expert in the art.

As used herein, the term “immunoreceptor” has its general meaning in the art and refers to a protein is expressed at the surface of leukocytes, which has two or more subunits and is capable of binding, specifically, to a given target molecule, preferably a protein. Such immunoreceptor is, for example, a B-cell receptor (BCR), which is expressed by B cells, a T-cell receptor (TCR) which is expressed by T cells or a Fc-receptor which is expressed by dendritic cells, monocytes, macrophages, neutrophils, eosinophils, mast cells, basophils, NK cells, platelets and Kupffer cells. The term “Fc-receptor”, short “FcR”, denotes a receptor that binds to an Fc-region. Fc receptors include FcγRI, FcγRII, and FcγRIII subclasses. FcγRII receptors include FcγRIIA (an “activating receptor”) and FcγRIIB (an “inhibiting receptor”). As used herein, the term “ITAM” has its general meaning in the art and is the acronym for Immunoreceptor Tyrosine-based Activation Motif. The ITAM motif is found in the cytoplasmic domain of the immunoreceptors. The immunoreceptors exert their inhibitory and activating signal through their ITAM motifs. According to the present invention when the ITAM motif confers an activation signalling the ITAM motif is named “ITAMa”. Conversely when the ITAM motif confers an inhibition signalling the ITAM motif is named “ITAMi”. The method of the present invention is thus particular suitable for determining whether an immunoreceptor is in an ITAMa or in an ITAMi configuration.

As used herein, the term “Fyn” has its general meaning in the art and refers to FYN proto-oncogene, Src family tyrosine kinase encoded by the FYN gene (Gene ID: 2534) and is also known as SLK; SYN; or p59-FYN. An exemplary human nucleic acid sequence for Fyn is accessible in GenBank under the access number NM_002037.5 (isoform a), NM_153047.3 (isoform b) or NM_153048.3 (isoform c). An exemplary human amino acid sequence for Fyn is accessible in GenBank under the accessible number NP_002028.1 (isoform a), NP_694592.1 (isoform b) or NP_694593.1 (isoform c). As used herein the term, pFynY528 indicates that the

Fyn protein is phosphorylated on the tyrosine residue at position 528 and the term pFynY417 indicates that the Fyn protein is phosphorylated on the tyrosine residue at position 417.

As used herein, the term “Lyn” has its general meaning in the art and refers to the LYN proto-oncogene, Src family tyrosine kinase encoded by the LYN gene (Gene ID: 4067) and is also known as JTK8; p53Lyn; p56Lyn. An exemplary human nucleic acid sequence for Lyn is accessibled in GenBank under the access number NM_001111097.2 (isoform B or NM_001111097.2 (isoform A). An exemplary human amino acid sequence for Lyn is accessible in GenBank under the accessible number NP_001104567.1 (isoform B), or NP_002341.1 (isoform A). As used herein, the term pLynY508 indicates that the Lyn protein is phosphorylated on the tyrosine residue at position 508 and the term 111411)(396/397 indicates that the Lyn protein is phosphorylated on the tyrosine residue at position 396 and/or 397.

As used herein the term “Lck” has its general meaning in the art and refers to the LCK proto-oncogene, Src family tyrosine kinase encoded by the LCK gene (Gene ID: 3932) and I s also known as LSK; YT16; IMD22; p561ck; pp581ck. An exemplary nucleic acid sequence for Lck is accessible in GenBank under the access number NM_001042771.2 or NM_005356.4. An exemplary amino acid sequence for Lck is accessible in GenBank under the access number NP_001036236.1 or NP_005347.3. As used herein, the term pLckY508 indicates that the Lck protein is phosphorylated on the tyrosine residue at position 508 and the term pLckY397 indicates that the Lck protein is phosphorylated on the tyrosine residue at position 397.

As used herein, the term “SHP-1” has its general meaning in the art and refers to the protein tyrosine phosphatase, non-receptor type 6 encoded by the PTPN6 gene (Gene ID 5777) and is also known as HCP; HCPH; SHPT; HPTP1C; PTP-1C; SHP-1L; or SH-PTP1. An exemplary nucleic acid sequence of SHP-1 is accessible in GenBank under the access number NM_002831.5. An exemplary nucleic acid sequence is accessible in GenBank under the access number NP_002822.2. As used herein, the term pSHP-1S591 indicates that the SHP-1 protein is phosphorylated in the serine at position 591. As used herein the term pSHP-1Y536 indicates that the SHP-1 protein is phosphorylated in tyrosine at position 536.

As used herein the term “PKCα” has its general meaning in the art and refers to protein kinase C alpha encoded by the gene PKCA (Gene ID: 5578) and is also known as AAG6; PKCA; PRKACA; PKC-alpha. An exemplary nucleic acid sequence for PKCα is accessible in GenBank under the access number NM_002737.2. An exemplary amino acid sequence for PKCα is accessible in GenBank under the access number NP_002728.1. As used herein the term pPKCα Thr638 indicates that the PKCalpha protein is phosphorylated in threonine at position 638.

In some embodiments, the method of the present invention comprises detection of the localization of Lyn, Lck, and Fyn.

In some embodiments, when the presence of Fyn is detected at the membrane it is concluded that the immunoreceptors are in an activating steady state of ITAM signalling.

In some embodiments, the method of the present invention comprises determining the phosphorylation profiling of Fyn, Lyn/Lck and SHP-1. In some embodiments, the method of the present invention comprises detecting the presence or absence of pSHP-1Y536, pSHP-1S591, pFyn^{Y528}, pFyn^{Y417}, pLyn/Lck^{Y396/397} or pLyn/Lck^{Y508}.

In some embodiments, the presence of pFyn^{Y417} and pSHP-1^{S591} indicates that the immunoreceptors are in an activating steady state of ITAM signalling. In some embodiments, the presence of pFyn^{Y417} and pSHP-1⁵⁹¹ and the absence of pFyn^{Y528} and pSHP-1^{Y436} indicates that the immunoreceptors are in an activating steady state of ITAM signalling.

In some embodiments, the presence of pLyn/Lck^{Y396/397} and pSHP-1^{Y536} indicates that the immunoreceptors are in a steady state inhibition of ITAMi signaling. In some embodiments, the presence of pLyn/Lck^{Y396/397} and pSHP-1^{Y536} and the absence of pLyn/Lck^{Y508} and pSHP-1^{S591} indicate that the immunoreceptors are in a steady state inhibition of ITAMi signaling.

In some embodiments, the method of the present invention comprises determining the phosphorylation profiling of PKCα. In some embodiments, the method of the present invention comprises detecting the absence or presence of pPKCα^{Thr638}. In some embodiments, the method of the present invention comprises detection the presence or absence of at least one marker selected from the group consisting of pSHP-1^{Y536} pFyn^{Y528}, pFyn^{Y417}, pSHP-1^{S591} and ppKCα^{Thr}638 . In some embodiments, the presence of pSHP-1⁵⁹¹ and pPKCα^{Thr638} indicates that the immunoreceptors are in an activating steady state of ITAM signalling. In some embodiments, the method of the present invention combines detection of the localization of Fyn and Lyn/Lck and the detection of at least one marker selected from the group consisting of pSHP-1^{Y536}, pFyn^{Y528}, pFyn^{Y417}, pSHP-1^{S591} and pPKCα^{Thr638}.

According to the invention, the detection of the marker is determined by any routine technique well known in the art. In some embodiments, the detection of the marker is determined by a flow cytometric and/or imagestream method.

As used herein, the term “flow cytometric method” refers to a technique for counting cells of interest, by suspending them in a stream of fluid and passing them through an electronic detection apparatus. Flow cytometric methods allow simultaneous multiparametric analysis of the physical and/or chemical parameters of up to thousands of events per second, such as fluorescent parameters. Modern flow cytometric instruments usually have multiple lasers and fluorescence detectors. As used herein, the “imagestream” refers to a technique for a flow cytometer that combines the speed, sensitivity, and phenotyping abilities of flow cytometry with the detailed imagery and functional insights of microscopy. This unique combination enables a broad range of applications that would be impossible using either technique alone. This instrument produces multiple high-resolution images of every cell directly in flow, including brightfield and darkfield (SSC), and up to 10 fluorescent markers with sensitivity exceeding conventional flow cytometers.

A common variation of flow cytometric techniques is to physically sort particles based on their properties, so as to purify or detect populations of interest, using “fluorescence-activated cell sorting”. As used herein, “fluorescence-activated cell sorting” (FACS) refers to a flow cytometric method for sorting a heterogeneous mixture of cells from a biological sample into two or more containers, one cell at a time, based upon the specific light scattering and fluorescent characteristics of each cell and provides fast, objective and quantitative recording of fluorescent signals from individual cells as well as physical separation of cells of particular interest. Accordingly, FACS can be used with the methods described herein to isolate and detect the population of cells of the present invention. For example, fluorescence activated cell sorting (FACS) may be therefore used. involves using a flow cytometer capable of simultaneous excitation and detection of multiple fluorophores, such as a BD Biosciences FACSCanto™ flow cytometer, used substantially according to the manufacturer's instructions. The cytometric systems may include a cytometric sample fluidic subsystem, as described below. In addition, the cytometric systems include a cytometer fluidically coupled to the cytometric sample fluidic subsystem. Systems of the present disclosure may include a number of additional components, such as data output devices, e.g., monitors, printers, and/or speakers, softwares (e.g. (Flowjo, Laluza . . . .), data input devices, e.g., interface ports, a mouse, a keyboard, etc., fluid handling components, power sources, etc.

Typically, the population of leukocytes is contacted with a panel of antibodies specific for the specific marker of interest. As used herein, the term “antibody” refers to an intact immunoglobulin or to a monoclonal or polyclonal antigen-binding fragment with the Fc (crystallizable fragment) region or FcR binding fragment of the Fc region. Antigen-binding fragments may be produced by recombinant DNA techniques or by enzymatic or chemical cleavage of intact antibodies. “Antigen-binding fragments” include, inter alia, Fab, Fab′, F(ab′)₂, Fv, dAb, and complementarity determining region (CDR) fragments, single -chain antibodies (scFv), single domain antibodies, chimeric antibodies, diabodies and polypeptides that contain at least a portion of an immunoglobulin that is sufficient to confer specific antigen binding to the polypeptide. The terms Fab, Fc, pFc′, F(ab′)₂ and Fv are employed with standard immunological meanings (Roitt, I. (1991) Essential Immunology, 7th Ed., (Blackwell Scientific Publications, Oxford)]. Such antibodies or antigen-binding fragments are available commercially from vendors such as R&D Systems, BD Biosciences, e-Biosciences, Biolegend, Proimmune and Miltenyi, or can be raised against these cell-surface markers by methods known to those skilled in the art.

In some embodiments, an agent that specifically bind to a marker of interest, such as an antibody or antigen-binding fragment, is labelled with a tag to facilitate the isolation and detection of population of cells of the interest. As used herein, the terms “label” or “tag” refer to a composition capable of producing a detectable signal indicative of the presence of a target, such as, the presence of a specific cell-surface marker in a biological sample. Suitable labels include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means. Non-limiting examples of fluorescent labels or tags for labeling the agents such as antibodies for use in the methods of invention include Hydroxycoumarin, Succinimidyl ester, Aminocoumarin, Succinimidyl ester, Methoxycoumarin, Succinimidyl ester, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, PerCPeFluor 710, PE-CF594, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5, Cy7, BV 785, BV711, BV421, BV605, BV510 or BV650.

The aforementioned assays may involve the binding of the antibodies to a solid support. The solid surface could be a microtitration plate coated with the antibodies. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In a preferred embodiment, fluorescent beads are those contained in TruCount(™) tubes, available from Becton Dickinson Biosciences, (San Jose, Calif.). In some embodiments, PBMC were stained for detection of cytokines production after stimulation in RPMI medium supplemented with 10% fetal bovine serum with PMA and ionomycin at 25 ng/mL and 1 μg/mL, respectively, in the presence of brefeldin A at 10 μg/mL for 6 hours at 37° C.

As being intra cellular located, the marker of the present invention are assessed by intracellular flow cytometry. Intracellular flow cytometry typically involves the permeabilization and fixation of the cells. Any convenient means of permeabilizing and fixing the cells may be used in practicing the methods. For example permeabilizing agent typically include saponin, methanol, Tween® 20, Triton X-100™.

In some embodiments, when the method of the present invention combines localization and detection, a flow imaging cytometry may be preferred. For example multispectral imaging flow cytometric analysis may be typically performed with an ImageStream™ instrument (Amnis Corporation, Seattle, Wash.). These methods of flow cytometery are described in the following commonly assigned patents: U.S. Pat. No. 6,249,341, issued on Jun. 19, 2001 and entitled “Imaging And Analyzing Parameters of Small Moving Objects Such As Cells;” U.S. Pat. No. 6,211,955 issued on Apr. 3, 2001, also entitled “Imaging And Analyzing Parameters of Small Moving Objects Such As Cells;” U.S. Pat. No. 6,473,176, issued on Oct. 29, 2002, also entitled “Imaging And Analyzing Parameters of Small Moving Objects Such As Cells;” U.S. Pat. No. 6,583,865, issued on Jun. 24, 2003, entitled “Alternative Detector Configuration And Mode of Operation of A Time Delay Integration Particle Analyzer;” U.S. patent application Ser. No. 09/989,031 entitled “Imaging And Analyzing Parameters of Small Moving Objects Such As Cells in Broad Flat Flow.”

In some embodiments, the method further comprises detecting the presence of absence of at least one phenotypic marker of B-Cell, T-cell, monocytes, neutrophils and basophils. Phenotypic markers of B cells are well known in the art an typically include CD5, CD38, CD19, CD40 and CD20 but also more specific markers such as CD24, CD21, CD27, CD1d and markers as functionally relevant for the impact of BCR signalling capacity such as CXCR4, CXCR5, CD62L and S1P1. Phenotypic markers of T-cell are well known in the art and typically include CD3, CD8, CD25, and Foxp3. Phenotypic markers of blood monocytes, neutrophils and basophils are well known in the art and typically include CD14; CD15, FcεRI/CCR3. In some embodiments, the method of the present invention further comprises detecting the presence or absence of at least one intracellular cytokine/chemokine. In some embodiments, the cytokine is a regulatory cytokines such as IL-10, TGF-β, IL-6 and granzyme.

The method of the present invention is particular suitable for the diagnosis of an inflammatory autoimmune disease. In particular the diagnostic method of the present invention comprises i) performing the method or the present invention in a blood sample obtained the patient and ii) concluding that the patient suffers from an inflammatory autoimmune disease when the it is concluded at step i) that the immunoreceptors of the leukocytes present in the blood sample are in an activating steady-state of ITAM signaling.

In some embodiments, the autoimmune inflammatory disease is selected from the group consisting of arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, juvenile-onset rheumatoid arthritis, osteoarthritis, arthritis chronica progrediente, arthritis deformans, polyarthritis chronica primaria, reactive arthritis, ankylosing spondylitis, inflammatory hyperproliferative skin diseases, psoriasis such as plaque psoriasis, gutatte psoriasis, pustular psoriasis, psoriasis of the nails, dermatitis including contact dermatitis, chronic contact dermatitis, allergic dermatitis, allergic contact dermatitis, dermatitis herpetiformis, atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma, systemic scleroderma, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (MPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus, optionally pemphigus vulgaris, pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoritis, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal gammopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl, and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, infertility due to antispermatozoan antibodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

In particular, when the patient is suspected of suffering from a renal inflammatory autoimmune disease, the method of the present invention is particularly suitable for determining whether a renal biopsy is required or not for confirming that diagnosis. Renal biopsy often exposes the patients to severe complications such as severe hematuria, arterial injury, requiring sometimes arterial embolization. In children, performing renal biopsy is often difficult. So the method of the invention offers a mean to avoid the renal biopsy if it is not necessary. Indeed, when it is concluded that the diagnosis of the disease is likely based on flow cytometer data, the physician can decide to avoid renal biopsy. In the opposite side, when this test is not in favor of the disease, the physician can decide performing a renal biopsy to clarify the diagnosis.

In some embodiments, the method of the present invention is particularly suitable for determining whether a patient suffering from an inflammatory autoimmune disease achieves a response with a treatment. In particular the monitoring method of the present invention comprises providing a blood sample of the patient after a period of treatment and concluding that the patient achieves a response when the immunoreceptor of the leukocytes present in the blood sample returns to an inhibiting steady state of ITAM signaling or concluding that the patient does not achieve a response when the immunoreceptor of the leukocytes present in the blood sample are maintained in their activating steady state of ITAM signaling.

Typically, the treatment involves use of immunosuppressive drug, corticosteroid and biotherapies for inhibiting the activity of an inflammatory cytokine such as TNF-alpha, IL-1beta, IL-6, IL-8, IL-17 . . . As used herein, the term “immunosuppressive drug” refers to any substance capable of producing an immunosuppressive effect, e.g., the prevention or diminution of the immune response.

Examples of immunosuppressive drugs include, without limitation, cyclosporine, thiopurine drugs such as azathioprine (AZA) and metabolites thereof; anti-metabolites such as methotrexate (MTX); sirolimus (rapamycin); temsirolimus; everolimus; tacrolimus (FK-506); FK-778; anti-lymphocyte globulin antibodies, anti-thymocyte globulin antibodies, anti-CD3 antibodies, anti-CD4 antibodies, and antibody-toxin conjugates; cyclosporine; mycophenolate; mizoribine monophosphate; scoparone; glatiramer acetate; metabolites thereof; pharmaceutically acceptable salts thereof; derivatives thereof; prodrugs thereof; and combinations thereof.

As used, the term “corticosteroids” has its general meaning in the art an refers to class of active ingredients having a hydrogenated cyclopentoperhydrophenanthrene ring system endowed with an anti-inflammatory activity. Corticosteroid drugs typically include cortisone, cortisol, hydrocortisone (11β,17-dihydroxy, 21-(phosphonooxy)-pregn-4-ene, 3,20-dione disodium), dihydroxycortisone, dexamethasone (21-(acetyloxy)-9-fluoro-1(3,17-dihydroxy-16μ-m-ethylpregna-1,4-diene-3,20-dione), and highly derivatized steroid drugs such as beconase (beclomethasone dipropionate, which is 9-chloro-11-β, 17,21, trihydroxy-16β-methylpregna-1,4 diene-3,20-dione 17,21-dipropionate). Other examples of corticosteroids include flunisolide, prednisone, prednisolone, methylprednisolone, triamcinolone, deflazacort and betamethasone. corticosteroids, for example, cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, and dexamethasone.

Typically the biotherapy consists in administering to the patient a therapeutically effective amount of an antibody or decoy receptor protein having specificity for the inflammatory cytokine or the receptor of the inflammatory cytokine. For example, the drug is an anti-TNFalpha drug. As used herein, the term “anti-TNFα drug” is intended to encompass agents including proteins, antibodies, antibody fragments, fusion proteins (e.g., Ig fusion proteins or Fc fusion proteins), multivalent binding proteins (e.g., DVD Ig), small molecule TNFα antagonists and similar naturally- or normaturally-occurring molecules, and/or recombinant and/or engineered forms thereof, that, directly or indirectly, inhibit TNFα activity, such as by inhibiting interaction of TNFα with a cell surface receptor for TNFα, inhibiting TNFα protein production, inhibiting TNFα gene expression, inhibiting TNFα secretion from cells, inhibiting TNFα receptor signaling or any other means resulting in decreased TNFα activity in a subject. The term “anti-TNFα drug” preferably includes agents which interfere with TNFα activity. Examples of anti-TNFα drugs include, without limitation, infliximab (REMICADE™, Johnson and Johnson), human anti-TNF monoclonal antibody adalimumab (D2E7/HUMIRA™, Abbott Laboratories), etanercept (ENBREL™, Amgen), certolizumab pegol (CIMZIA®, UCB, Inc.), golimumab (SIMPONI®; CNTO 148), CDP 571 (Celltech), CDP 870 (Celltech), as well as other compounds which inhibit TNFα activity, such that when administered to a subject in which TNFα activity is detrimental, the disorder (i.e. acute severe colitis) could be treated.

A further object of the invention relates to kit comprising means for performing the method of the present invention. Typically, the kit comprises means for detection of the presence or absence of the markers of interest. In some embodiments, said means are antibodies as described above. In some embodiments, these antibodies are labelled as described above. Typically, the kits described above will also comprise one or more other containers, containing for example, wash reagents, and/or other reagents capable of quantitatively detecting the presence of bound antibodies. The kit also contains agents suitable for performing intracellular flow cytometry such as agents for permeabilization and fixation of cells. Typically compartmentalised kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept the blood sample, a container which contains the antibody(s) used in the assay, containers which contain wash reagents (such as phosphate buffered saline, Tris-buffers, and like), and containers which contain the detection reagent.

The invention will be further illustrated by the following figures and examples. However, these examples and figures should not be interpreted in any way as limiting the scope of the present invention.

### FIGURES

FIG. 1. Differential regulation of FcγRIIA-ITAM signals by SFK Lyn and Fyn. (A) For ITAMi signaling, monocytic THP-1-CD14⁺-FcγRIIA⁺ cell line (transfected with siRNA as indicated) was incubated for the indicated time periods with 10 μg/mL of IV.3 anti-FcγRIIA F(ab′)₂ fragments at 37° C. Moreover, FcγRIIB was not detected on THP-1-CD14⁺-FcγRIIA⁺ as described (Ben Mkaddem et al., 2014). For ITAM signaling, cells were incubated with 10 μg/ml of indicated F(ab′)₂ at 4° C. followed by an anti-κ light chain F(ab′)₂ at 37° C. for indicated time points. After immunoprecipitation (IP), immunoblots (IB) were performed with indicated Abs. Quantification of the indicated band using ImageJ software relative to total corresponding protein levels in cell lysates (see fig S1) is indicated at the bottom of each panel, representing one out of at least three experiments. (B) Modulation of LPS-mediated IL-8 production by Lyn and Fyn during FcγRIIA-ITAMi induction. THP-1-CD14⁺-FcγRIIA⁺ cells transfected with indicated siRNAs were stimulated for indicated time points to induce either ITAMi or ITAM signals followed by stimulation with LPS (10 ng/ml) for 1 hour. Then, supernatant was collected for cytokine measurement. (C) Modulation of IL-8 production by Lyn and Fyn during FcγRIIA-ITAM induction for 18 hours. Data are presented as the mean±s.e.m. * **P<0.001; Student's unpaired t-test.

FIG. 2. Differential regulation of FcαRI-ITAM signals by Lyn and Fyn. THP-1 cells were transfected by siRNA as indicated. For ITAMi, cells were stimulated with 10 μg/mL of anti-FcαRI F(ab′)₂ at 37° C. for indicated times. For ITAM, cells were incubated for 30 min with 10 μg/ml of anti-FcαRI F(ab′)₂ at 4° C. followed by an anti-kappa light chain F(ab′)₂ at 37° C. After FcαRI immunoprecipitation (IP), western blots were performed with the indicated Abs. Quantification of the indicated band using ImageJ software relative to total corresponding protein levels in cell lysates is shown at the bottom of each panel, representing one out of at least three experiments.

FIG. 3. Differential regulation of BCR- or TCR-ITAM signals by SFK Lyn/Lck and Fyn. (A) For ITAMi signaling, Ramos B cells (transfected with siRNA as indicated) were incubated for the indicated time periods with 10 μg/mL of anti-CD79a Ab F(ab′)₂ fragments at 37° C. For ITAM signaling, cells were incubated with 10 μg/ml of anti-CD79aAb F(ab′)₂ fragments at 4° C. followed by an anti-κ light chain F(ab′)₂ at 37° C. for indicated time points. After immunoprecipitation (IP), immunoblots (IB) were performed with indicated Abs. Quantification of the indicated band using ImageJ software relative to total corresponding protein levels in cell lysates is shown at the bottom of each panel, representing one out of at least three experiments. (B) For ITAMi signaling, Jurkat T cell cell lines (transfected with siRNAs as indicated) were incubated for the indicated time periods with 10 μg/mL of anti-CD3 Ab F(ab′)₂ fragments at 37° C. For ITAM signaling, cells were incubated with 10 μg/ml of anti-CD3 Ab F(ab′)₂ fragments at 4° C. followed by an anti-κ light chain F(ab′)₂ at 37° C. for indicated time points. After immunoprecipitation (IP), immunoblots (IB) were performed with indicated Abs. Quantification of the indicated band using ImageJ software relative to total corresponding protein levels in cell lysates is shown at the bottom of each panel, representing one out of at least three experiments.

FIG. 4. SFK modulates cytokine production during BCR- or TCR-ITAM signals. (A) Modulation of Pam3csk4-mediated IL-8 production by Lyn and Fyn during BCR-ITAMi signaling. Ramos B cells (transfected with indicated siRNAs) were stimulated for 30 to induce ITAMi signal followed by stimulation with Pam3csk4 (1 μg/ml) for 6 hours. Then, supernatant was collected for cytokine measurement. (B) Modulation of IL-8 production dependent on BCR-induced ITAM signaling by Lck and Fyn. Ramos B cells (transfected by siRNA as indicated) were first incubated with 10 μg/mL of the indicated F(ab′)₂ at 4° C. for 30 min followed by anti-κ light chain F(ab′)₂ fragments at 37° C. for 6 hours. Supernatant were then collected for cytokine measurement using ELISA. (C) Modulation of FlageIlin-mediated IL-2 production by Lck and Fyn during TCR-ITAMi signaling. Jurkat cells (transfected with indicated siRNAs) were stimulated for 30 min to induce ITAMi signal followed by stimulation with FlageIlin (5 μg/ml) for 6 hours. Then, supernatant was collected for cytokine measurement. (D) Modulation of IL-2 production dependent on TCR-induced ITAM signaling by Lck and Fyn. Jurkat cells (transfected by siRNAs as indicated) were first incubated with 10 μg/mL of the indicated F(ab′)₂ at 4° C. for 30 min followed by anti-κ light chain F(ab′)₂ fragments at 37° C. for 18 hours. Supernatant were then collected for cytokine measurement using ELISA. Data are presented as the mean±s.e.m. n=3. ***/3<0.001; Student's unpaired t-test.

FIG. 5. SFK-mediated differential regulation of TCR-mediated ITAM signals and their effects on FlageIlin-dependent IL-2 production. For ITAMi, Jurkat cells (transfected by siRNA as indicated) were first incubated with 10 μg/mL of indicated F(ab′)₂ fragments at 37° C. for 30 min followed by stimulation with FlageIlin (5 μg/ml) for 6 hours. For ITAM, Jurkat cells (transfected by siRNA as indicated) were incubated with anti-CD3 F(ab′)₂ fragments plus anti-κ F(ab′)₂ fragments for 6 hours. PMA and ionomycin were used as positive control for IL-2 production. (A) Representative contour plots of intracellular IL-2 staining on fixed/permeabilized Jurkat cells. (B) Quantification of IL-2′ cells after different stimuli from three independent experiments, ***P<0.001; Student's unpaired t-test. ns, non significant. Data are presented as the mean±s.e.m. n=3.

### EXAMPLE 1

Experimental Procedures

Study Subjects

Fourteen individuals (eight healthy individuals and six patients) were studied. The SLE group was composed of 6 patients attending or referred to the Bichat's Hospital specialist nephrology unit between July 2014 and January 2016 meeting at least four ACR systemic lupus erythematosus criteria (Tan et al., 1982) presenting with active disease with nephritis proven by kidney biopsy (2 at class IV and 4 at class V) and in whom peripheral blood by venepuncture was obtained immediately prior to immunosuppressive therapy administration. All patients were female with age varying between 25 and 42. Ethical approval for this study was obtained from the Bichat Hospital Local Research. Ethics Committee and informed consent was obtained from all subjects enrolled.

Cells and Reagents

Human blood samples (12 ml) were first submitted to red cell lysis and pellets of 107 leukocytes were subjected RIPA buffer treatment (see below). BMM from 6- to 8-week-old mice were obtained after a 7-day culture with M-CSF (R&D systems). THP-1 and THP1-Fc γ RIIA-R131⁺-CD14⁺ cell lines (kindly provided by Novimmune) (Shang et al., 2014) were maintained in RPMI-1640, 10% FCS and 50 μM β-mercaptoethanol or supplemented with 200 μg/ml Zeocin, 10 μg/ml blasticidin and 2 μg/ml puromycin (Invitrogen, France). Jurkat and Ramos human cell lines were maintained in RPMI-1640 supplemented with 10% FCS and antibiotics. FCS was removed from the culture medium immediately before stimulation as described (Akhade and Qadri, 2015). Mouse mAbs antihuman FcγRII (clones IV.3 and AT-10), anti-human CD3 (clone HIT-3a) or anti-human CD79a(clone ZL7-4) were purchased from Santa Cruz and used in their F(ab′)₂ fragment forms. Mouse mAb anti-human FcαRI (clone A77) and irrelevant control mAb (320) were purified in-house and were used as F(ab′)₂, as previously described (Ben Mkaddem et al., 2014; Pasquier et al., 2005). For biochemical studies, rabbit anti-Syk, anti-Zap70, antiSHP-1, anti-Lyn, anti-Lck, anti-Fyn, anti-ERK (all from Santa Cruz Biotechnology), anti-SHP1 (phospho-Y536) (ECM Biosciences), and anti-SHP1 (phospho-S591) (Abcam) were used. Anti-pERK, anti-pAKT, anti-pPKCα, anti-AKT and anti-PKCα were from Cell Signaling.

Cell Stimulation

For ITAMi signaling, 5×10⁶ of monocytic cell lines (THP-1-CD14⁺-FcγRIIA⁺ or THP-1-CD14⁺), Jurkat T cell and Ramos B cell lines (transfected with different siRNA) were incubated for 30 min with 10 μg/mL of anti-FcγRIIA (clone IV.3), anti-CD3 (clone HIT-3a) or anti-CD79a(clone ZL7-4) F(ab′)₂ fragments at 37° C., respectively. Cells were then incubated with or without LPS (10 ng/ml) and FlageIlin (5 μg/ml) as described (Pinheiro da Silva et al., 2007) or Pam3csk4 (1 μg/ml) for 1 hour for monocytic cell lines and 18 hours for Jurkat and Ramos. For ITAM signaling, cells were incubated with 10 μg/ml of anti-FcγRIIA (clone IV.3), anti-CD3 (clone HIT-3a) or anti-CD79a(clone ZL7-4) F(ab′)₂ at 4° C. followed by an anti-κ light chain F(ab′)₂ at 37° C. for 18 h for cytokine measurement or 6 hours for intracellular IL-2 staining.

Analysis of Intracellular Cytokine Staining

After washing, Jurkat cells were incubated with or without anti-CD3 F(ab′)₂ fragment or with preformed complexes of anti-CD3 F(ab′)₂ plus anti-kappa F(ab′)₂ fragments. Cells were then stimulated or not with flageIlin (1 μg) for 6 hours. PMA (40 nM) and ionomycin (1 nM) were used as positive stimuli for 6 hours. Brefeldin A was added after 2 hours stimulation and maintained for 4 hours. The stimulation was stopped by adding 1 ml cold PBS. Intracellular cytokine staining was performed on fixed/permeabilized cells in residual permeabilization wash buffer (Biolegend, USA) using a conjugated antibody (anti-IL-2 PE or appropriate isotype control) for 20 min in the dark at room temperature as described (Magalhaes et al., 2015). Data acquisition was performed using a BD Biosciences LSR Fortessa cytometer, and results were analyzed using FlowJo analysis software (Tree Star).

Immunoprecipitation and Immunoblotting

Cells (5×10⁶ to 10⁷) were solubilized in RIPA lysis buffer containing 1% Nonidet P-40/0.1% sodium dodecyl sulfate (SDS) as described⁸. For immunoprecipitation, cell lysates were incubated with 2 μg/ml of IV.3 anti-FcγRIIA, A77 anti-FcαRI, HIT-3a anti-CD3 or ZL7-4 anti-CD79amAbs and immunoprecipitated overnight at 4° C. with Protein G-Sepharose (GE Healthcare). Samples were resolved by SDS polyacrylamide gel electrophoresis (10%), transferred to nitrocellulose membranes and immunoblotted with rabbit antibodies followed by goat antirabbit IgG (GE Healthcare) coupled to horseradish peroxidase. Membranes were developed by enhanced chemical luminescence treatment (Amersham Biosciences).

Enzyme-Linked Immunosorbent Assay (ELISA)

IL-8 and IL-2 were measured in the supernatants of stimulated cells using ELISA kits (R&D Systems) according to the manufacturer's instructions.

Histological Analysis

For immunofluorescence studies, frozen kidney sections were incubated in blocking buffer (PBS, 0.3% saponine, 1% BSA) for 30 min, and then with anti-phospho SHP-1^{Y536} AF488, SHP-1^{S591} AF647 (Bioss, 1/50 dilution), and anti-Phaloidin AF568 (life technology,)/100 dilution) for 2 hrs. Fluorescence was detected by confocal laser scanning microscopy (CLSM-510-META, Zeiss).

siRNA Transfections

Experiments were performed using predesigned HP GenomeWide (Qiagen, Courtaboeuf, France) Single strand sense and antisense RNA nucleotides were annealed to generate an RNA duplex according to the manufacturer's instructions. Cell lines were incubated with 5 to 10 nM of each siRNA tested and 2 μl of Lipofectamine® RNAiMAX prepared according to the manufacturer's instructions (Invitrogen, Saint Aubin, France) for 48 or 72 hrs at 37° C. before use. BMMs were incubated at day 4 during M-CSF-induced differentiation with 20 nM of each siRNA tested and 2 μl of Lipofectamine® RNAiMAX prepared according to the manufacturer's instructions (Invitrogen, Saint Aubin, France) for 48 hours at 37° C. before use.

Statistical Analysis

Statistical analyses were performed using GraphPad Prism software http://www.graphpad.com/scientific-software/prism/. All data were expressed as mean±SEM. Statistical significance between two groups was examined by the Student's t-test or the Mann-Whitney test, while the one-way and two-way analysis of variance (ANOVA) with Bonferroni's, Holm-Sidak's or Newman-Keuls multiple comparisons test were used to evaluate multiple groups. P-values of 0.05 were considered significant; values less than 0.05 are indicated in the figure legends.

Results:

Distinct SFKs Differentially Regulate Immunoreceptor-ITAM Signals.

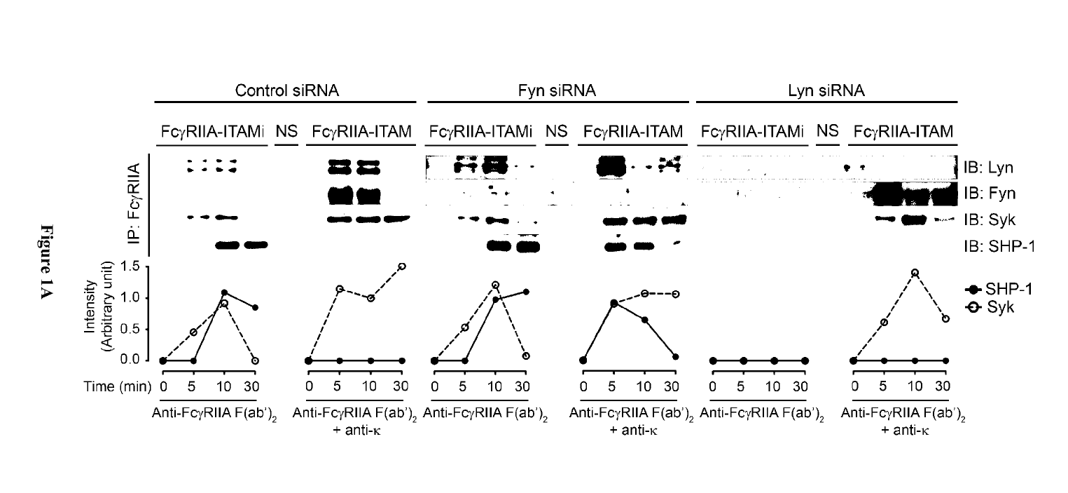

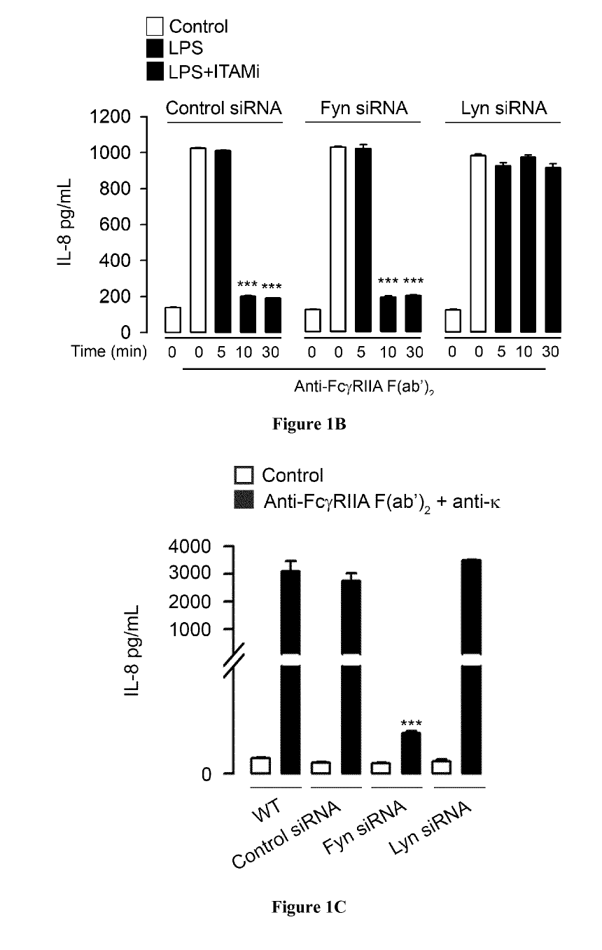

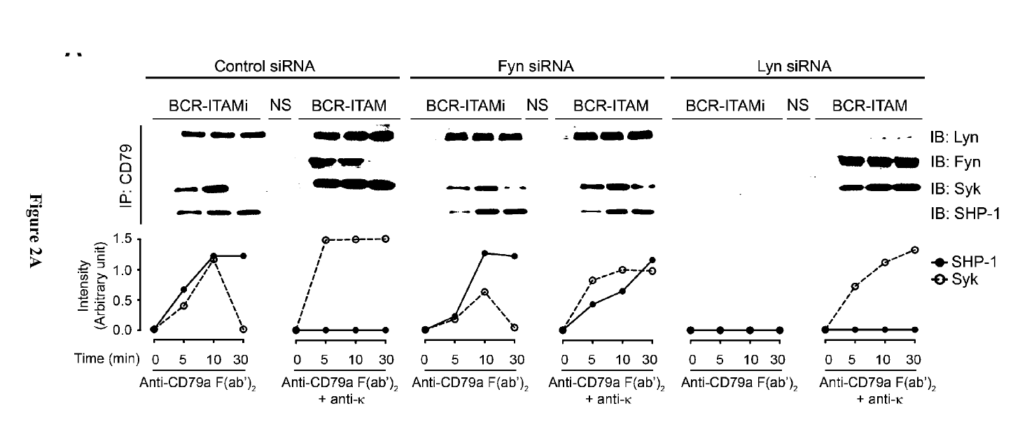

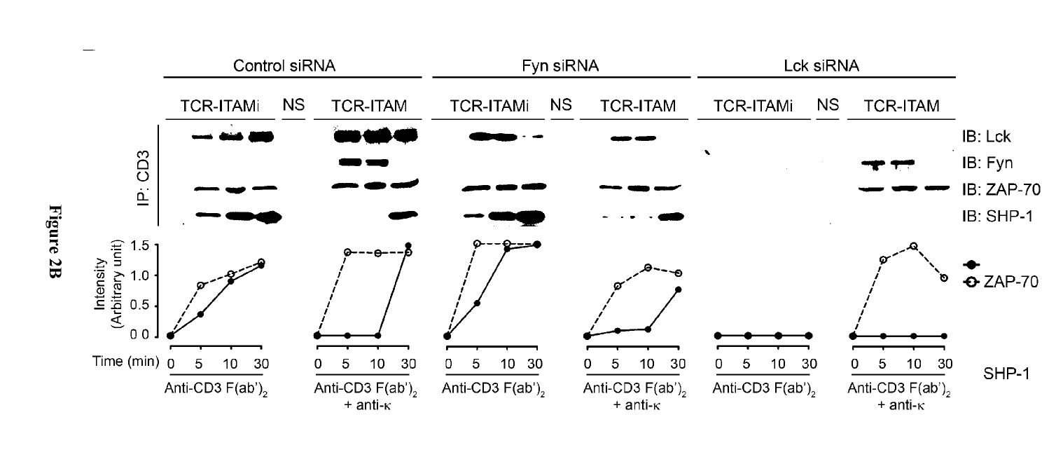

In order to address whether SFKs play a role in the switch between ITAMi or ITAM signaling, we first downregulated Fyn or Lyn expression in representative human monocytic cell lines by siRNA strategy. The cells were then stimulated for ITAMi induction by divalent targeting, or for ITAM signals by multivalent crosslinking of FcγRIIA or FcαRI as described previously (Ben Mkaddem et al., 2014; Pasquier et al., 2005). The ITAMi molecular signature was characterized by transient Syk recruitment followed by stable and prolonged SHP-1 recruitment, which required the presence of Lyn (but not Fyn). In contrast, multivalent crosslinking of these receptors induced an ITAM molecular signature with a stable recruitment of Syk (but not SHP1), requiring the recruitment of Fyn (FIGS. 1A and 2A, left panel). Fyn silencing had no effect on ITAMi signaling but reversed the ITAM to an ITAMi signature by the recruitment of SHP-1 to the receptors despite the multivalent crosslinking (FIGS. 1A and 2A, middle panel), whereas Lyn silencing completely abolished ITAMi signaling without affecting the ITAM signal (FIGS. 1A and 2A, right panel). Functional consequences of individual SFK silencing were then evaluated. Lyn but not Fyn was essential for the ITAMi inhibitory signals observed in FcR-mediated inhibition of LPS-mediated IL-8 production

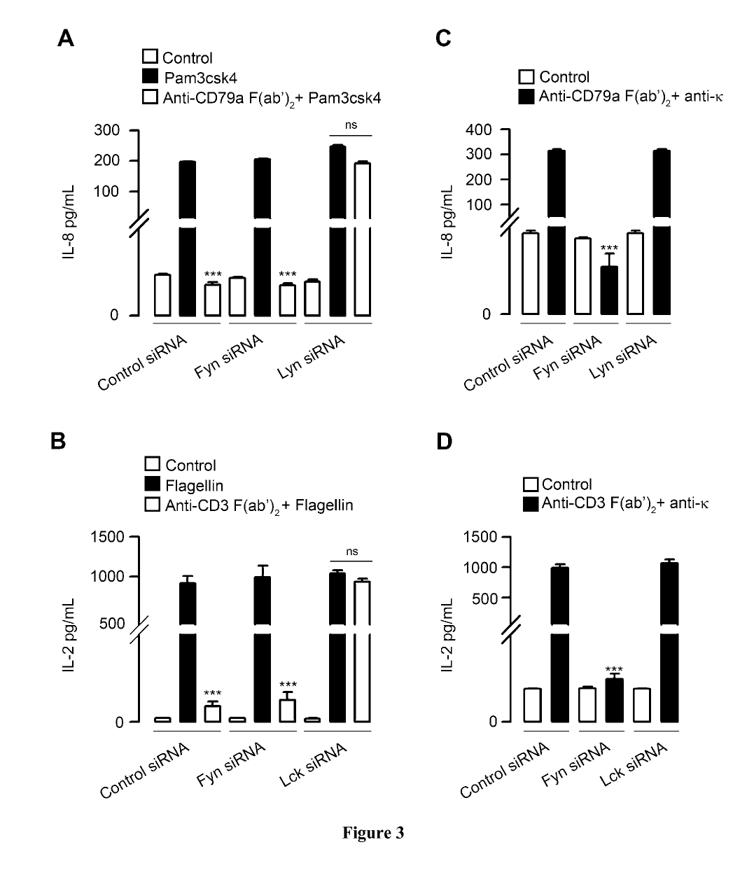

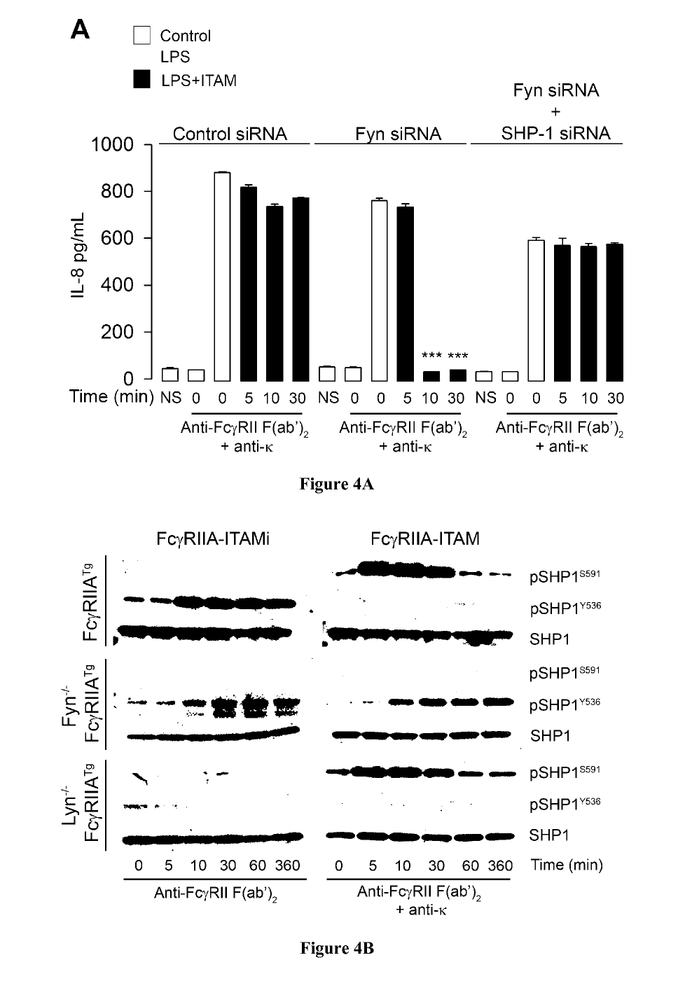

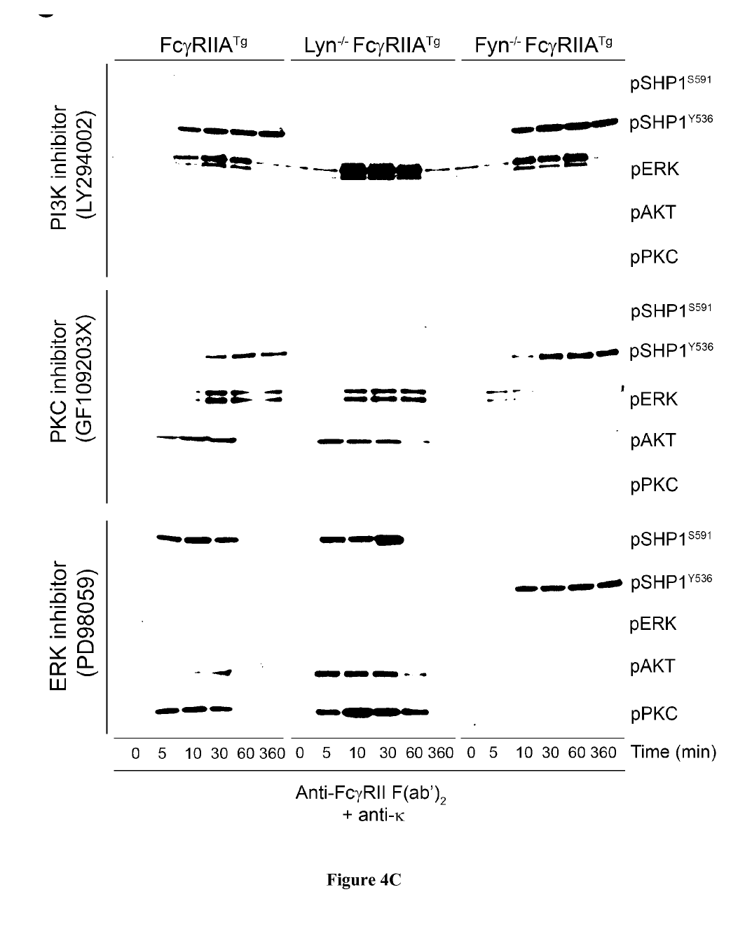

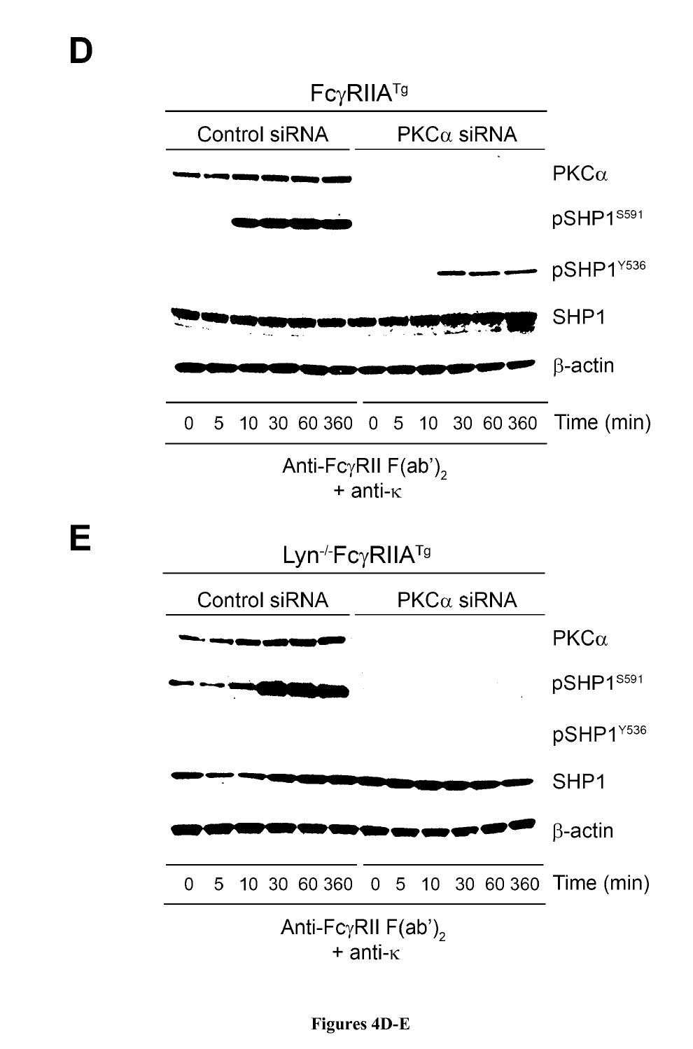

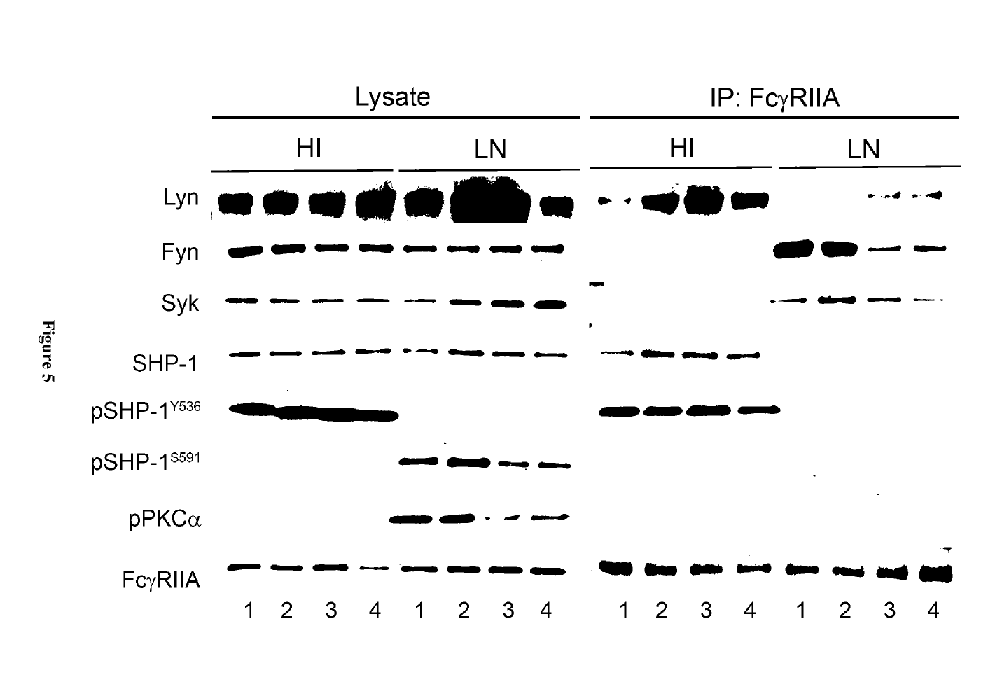

(FIG. 1B and 2B). By contrast, Fyn but not Lyn was essential for ITAM-mediated cell activation as measured by IL-8 production after multivalent engagement of FcγRIIA (FIG. 1C) or FcαRI (FIG. 2C). These results reveal opposing roles of the SFK Fyn and Lyn, transmitting, respectively, activating or inhibitory signals depending on the type of ligand interaction. To investigate whether other ITAM-bearing receptors could also deliver such opposite signals, divalent or multivalent targeting of BCR and TCR were performed using anti-CD79a or anti-CD3 F(ab′)₂ fragments alone or complexed with anti-κ light chains in representative lymphocytic cell lines. Similarly to FcRs, divalent targeting of both BCR and TCR resulted in typical ITAMi signatures, while multivalent crosslinking led to the expected ITAM signature (FIGS. 3A, 3B). Moreover, TCR-Fyn dissociation was linked to SHP-1 recruitment suggesting that Fyn could compete with SHP-1 recruitment (FIG. 3B, left panel). These results support that BCR- or TCR-mediated ITAMi signals like for FcRs, require Lyn or Lck , whereas Fyn was essential for activating ITAM signals. We next assessed whether BCR and TCR following ITAMi-dependent low valency engagement were also capable to inhibit signaling responses of heterologous receptors by evaluating TLR-induced cytokine production after their stimulation. The results clearly show that BCR- or TCR-divalent targeting was able to inhibit cytokine production induced by heterologous receptors requiring Lyn or Lck (FIGS. 4A and 4B). By contrast, Fyn was required for autologous cytokine production after BCR and TCR multivalent engagement (FIGS. 4C and 4D). These results were validated by intracellular IL-2 staining of siRNA-transfected cells of the T cell line Jurkat (FIG. 5). These results demonstrate for the first time that anergic ITAMi signaling of BCR and TCR, like for FcRs is able to induce an inhibitory crosstalk with other receptors dampening inflammatory responses. They shed new light on previous findings using weakly binding ligands or antibodies that trigger negative signals by BCR and TCR (Bolt et al., 1993; Feinerman et al., 2008; Kraus et al., 2001; Stefanova et al., 2003).

SFK mediate differential SHP-1 Phosphorylation controlling its activity To address the mechanism by which Lyn/Lck regulate ITAMi signalling, we exploited our findings that Fyn deletion reverses ITAM to ITAMi signals to explore a possible link between SHP-1 and Fyn under ITAM configuration. We confirmed that Fyn silencing readily inhibited cytokine production after multivalent targeting. Interestingly however, the silencing of both SHP-1 and Fyn restored LPS-mediated IL-8 production (data not shown), indicating that Fyn abrogates an SHP-1-mediated inhibitory signal. Previously, phosphorylation of SHP-1 on Y536 and S591 residues has been associated with respectively its activation and inactivation (Jones et al., 2004; Liu et al., 2007). Hence we analyzed whether Lyn and Fyn could control SHP-1 function through differential phosphorylation of SHP-1. Simulation of bone marrow-derived macrophages (BMM) from FcγRIIA^{Tg} (R131 isoform) of mice (Tan Sardjono et al., 2005) under ITAMi conditions showed that Lyn induced activating SHP-1 Y536 phosphorylation. By contrast, multivalent ITAM stimulation induced a Fyn dependent SHP-1 S591 phosphorylation, (data not shown). Moreover, under these conditions the absence of Fyn resulted in Lyn-dependent Y536 phosphorylation of SHP-1, thus mimicking an ITAMi signal (data not shown). To understand how Fyn, a tyrosine kinase, could promote SHP-1 serine phosphorylation, we performed FcγRIIA-ITAM stimulation in the presence or absence of ERK, PKC and PI3K inhibitors (Daniels et al., 2010; Parravicini et al., 2002). Whereas PI3K and PKC inhibitors completely blocked both SHP-1^{S591} and PKC phosphorylation, the ERK inhibitor had no effect (data not shown). In addition, both PI3K and PKC inhibitors favoured SHP-1^{Y536} phosphorylation under conditions of FcγRIIA-ITAM signaling, and this preference required the presence of Lyn (data not shown). Interestingly, silencing of the PKCα isoform also abrogated SHP-1^{S591} phosphorylation under conditions of FcγRIIA multimeric aggregation but favoured Lyn dependent SHP-1^{Y536} phosphorylation (data not shown), which was Lyn-dependent (data not shown). Together, these results indicate that during ITAM signaling, Fyn inactivates SHP-1 through phosphorylation of the S591 involving a PI3K-PKCα axis, thereby blocking its activation by Lyn.

SHP-1^{S591} Phosphorylation and Fyn Recruitment are Linked to Severe Nephritis in Lupus Patients

To examine whether Fyn-mediated inhibitory SHP-1^{S591} phosphorylation were associated with immune complex-mediated disease via the FcγRIIA in patients with a given inflammatory disease, we analyze blood leukocytes from untreated patients with lupus nephritis at different stages of renal involvement morphologically classified as class IV-A (severe nephritis with immune deposits and leukocyte infiltration) and pure class V (membranous immune deposits only). pSHP-1^{S591} and pPKCα were exclusively observed in patient cell lysates, but not associated with FcγRIIA (data not shown). Consistent with the role of Lyn/SHP-1 axis in homeostasis, Lyn and pSHP-1^{Y536} were strongly associated with FcγRIIA in healthy individuals underlining the inhibitory homeostatic phenotype, whereas Fyn and Syk were exclusively associated with FcγRIIA in patients highlighting the deleterious role of ITAM signalling in inflammatory disease. These results identified pSHP-1^{S591} and Fyn recruitment as novel biomarkers for severe lupus nephritis

### REFERENCES

Throughout this application, various references describe the state of the art to which this invention pertains. The disclosures of these references are hereby incorporated by reference into the present disclosure.

Abram, C. L., and Lowell, C. A. (2007). The expanding role for ITAM-based signaling pathways in immune cells. Sci STKE 2007, re2.

Abram, C. L., Roberge, G. L., Pao, L. I., Neel, B. G., and Lowell, C. A. (2013). Distinct roles for neutrophils and dendritic cells in inflammation and autoimmunity in motheaten mice. Immunity 38, 489-501.

Akhade, A. S., and Qadri, A. (2015). T-cell receptor activation of human CD4(+) T cells shifts the innate TLR response from CXCL8(hi) IFN-gamma(null) to CXCL8(lo) IFN-gamma(hi). Eur J Immunol 45, 2628-2637.

Aloulou, M., Ben Mkaddem, S., Biarnes-Pelicot, M., Boussetta, T., Souchet, H., Rossato, E., Benhamou, M., Crestani, B., Zhu, Z., Blank, U., et al. (2012). IgG1 and IVIg induce inhibitory ITAM signaling through FcgammaRIII controlling inflammatory responses. Blood 119, 3084-3096.

Appleby, M. W., Gross, J. A., Cooke, M. P., Levin, S. D., Qian, X., and Perlmutter, R. M. (1992). Defective T cell receptor signaling in mice lacking the thymic isoform of p59fyn. Cell 70, 751-763.

Ben Mkaddem, S., Hayem, G., Jonsson, F., Rossato, E., Boedec, E., Boussetta, T., El Benna, J., Launay, P., Goujon, J. M., Benhamou, M., et al. (2014). Shifting FcgammaRIIA-ITAM from activation to inhibitory configuration ameliorates arthritis. J Clin Invest 124, 3945-3959.

Bezbradica, J. S., and Medzhitov, R. (2012). Role of ITAM signaling module in signal integration. Curr Opin Immunol 24, 58-66.

Blank, U., Launay, P., Benhamou, M., and Monteiro, R. C. (2009) Inhibitory ITAMs as novel regulators of immunity. Immunol Rev 232, 59-71.

Bolt, S., Routledge, E., Lloyd, I., Chatenoud, L., Pope, H., Gorman, S. D., Clark, M., and Waldmann, H. (1993). The generation of a humanized, non-mitogenic CD3 monoclonal antibody which retains in vitro immunosuppressive properties. Eur J Immunol 23, 403-411.

Charles, N., Watford, W. T., Ramos, H. L., Hellman, L., Oettgen, H. C., Gomez, G., Ryan, J. J., O'Shea, J. J., and Rivera, J. (2009). Lyn kinase controls basophil GATA-3 transcription factor expression and induction of Th2 cell differentiation. Immunity 30, 533-543.

Croker, B. A., Lawson, B. R., Rutschmann, S., Berger, M., Eidenschenk, C., Blasius, A. L., Moresco, E. M., Sovath, S., Cengia, L., Shultz, L. D., et al. (2008). Inflammation and autoimmunity caused by a SHP1 mutation depend on IL-1, MyD88, and a microbial trigger. Proc Natl Acad Sci U S A 105, 15028-15033.

Daniels, A. B., Worth, R. G., Dickstein, R. J., Dickstein, J. S., Kim-Han, T. H., Kim, M. K., and Schreiber, A. D. (2010). Analysis of FcgammaRIIA cytoplasmic tail requirements in signaling for serotonin secretion: evidence for an ITAM-dependent, PI3K-dependent pathway. Scand J Immunol 71, 232-239.

Feinerman, O., Veiga, J., Dorfman, J. R., Germain, R. N., and Altan-Bonnet, G. (2008). Variability and robustness in T cell activation from regulated heterogeneity in protein levels. Science 321, 1081-1084.

Gauld, S. B., and Cambier, J. C. (2004). Src-family kinases in B-cell development and signaling. Oncogene 23, 8001-8006.

Getahun, A., Beavers, N. A., Larson, S. R., Shlomchik, M. J., and Cambier, J. C. (2016). Continuous inhibitory signaling by both SHP-1 and SHIP-1 pathways is required to maintain unresponsiveness of anergic B cells. J Exp Med.

Getahun, A., and Cambier, J. C. (2015). Of ITIMs, ITAMs, and ITAMis: revisiting immunoglobulin Fc receptor signaling. Immunol Rev 268, 66-73.

Hamerman, J. A., and Lanier, L. L. (2006) Inhibition of immune responses by ITAM-bearing receptors. Sci STKE 2006, rel.

Hibbs, M. L., Tarlinton, D. M., Armes, J., Grail, D., Hodgson, G., Maglitto, R., Stacker, S. A., and Dunn, A. R. (1995). Multiple defects in the immune system of Lyn-deficient mice, culminating in autoimmune disease. Cell 83, 301-311.

Hogarth, P. M. (2002). Fc receptors are major mediators of antibody based inflammation in autoimmunity. Curr Opin Immunol 14, 798-802.

Holdorf, A. D., Green, J. M., Levin, S. D., Denny, M. F., Straus, D. B., Link, V., Changelian, P. S., Allen, P. M., and Shaw, A. S. (1999). Proline residues in CD28 and the Src homology (SH)3 domain of Lck are required for T cell costimulation. J Exp Med 190, 375-384.

Iwashima, M., Irving, B. A., van Oers, N. S., Chan, A. C., and Weiss, A. (1994). Sequential interactions of the TCR with two distinct cytoplasmic tyrosine kinases. Science 263, 1136-1139.

Janas, M. L., Hodgkin, P., Hibbs, M., and Tarlinton, D. (1999). Genetic evidence for Lyn as a negative regulator of IL-4 signaling. J Immunol 163, 4192-4198.

Johnson, D. J., Pao, L. I., Dhanji, S., Murakami, K., Ohashi, P. S., and Neel, B. G. (2013). Shp1 regulates T cell homeostasis by limiting IL-4 signals. J Exp Med 210, 1419-1431.

Jones, M. L., Craik, J. D., Gibbins, J. M., and Poole, A. W. (2004). Regulation of SHP-1 tyrosine phosphatase in human platelets by serine phosphorylation at its C terminus. J Biol Chem 279, 40475-40483.

Kanamaru, Y., Pfirsch, S., Aloulou, M., Vrtovsnik, F., Essig, M., Loirat, C., Deschenes, G., Guerin-Marchand, C., Blank, U., and Monteiro, R. C. (2008). Inhibitory ITAM signaling by Fc alpha RI-FcR gamma chain controls multiple activating responses and prevents renal inflammation. J Immunol 180, 2669-2678.

Khalil, A. M., Cambier, J. C., and Shlomchik, M. J. (2012). B cell receptor signal transduction in the GC is short-circuited by high phosphatase activity. Science 336, 1178-1181.

Kraus, M., Pao, L. I., Reichlin, A., Hu, Y., Canono, B., Cambier, J. C., Nussenzweig, M. C., and Rajewsky, K. (2001). Interference with immunoglobulin (Ig)alpha immunoreceptor tyrosine-based activation motif (ITAM) phosphorylation modulates or blocks B cell development, depending on the availability of an Igbeta cytoplasmic tail. J Exp Med 194, 455-469.

Liu, Y., Kruhlak, M. J., Hao, J. J., and Shaw, S. (2007). Rapid T cell receptor-mediated SHP-1 S591 phosphorylation regulates SHP-1 cellular localization and phosphatase activity. J Leukoc Biol 82, 742-751.

Luo, K. X., and Sefton, B. M. (1990). Cross-linking of T-cell surface molecules CD4 and CD8 stimulates phosphorylation of the lck tyrosine protein kinase at the autophosphorylation site. Mol Cell Biol 10, 5305-5313.

Magalhaes, I., Pingris, K., Poitou, C., Bessoles, S., Venteclef, N., Kiaf, B., Beaudoin, L., Da Silva, J., Allatif, O., Rossjohn, J., et al. (2015). Mucosal-associated invariant T cell alterations in obese and type 2 diabetic patients. J Clin Invest 125, 1752-1762.

Mkaddem, S. B., Christou, I., Rossato, E., Berthelot, L., Lehuen, A., and Monteiro, R. C. (2014). IgA, IgA receptors, and their anti-inflammatory properties. Curr Top Microbiol Immunol 382, 221-235.

Nishizumi, H., Taniuchi, I., Yamanashi, Y., Kitamura, D., Ilic, D., Mori, S., Watanabe, T., and Yamamoto, T. (1995). Impaired proliferation of peripheral B cells and indication of autoimmune disease in lyn-deficient mice. Immunity 3, 549-560.

Packard, T. A., and Cambier, J. C. (2013). B lymphocyte antigen receptor signaling: initiation, amplification, and regulation. F1000Prime Rep 5, 40.

Palacios, E. H., and Weiss, A. (2004). Function of the Src-family kinases, Lck and Fyn, in T-cell development and activation. Oncogene 23, 7990-8000.

Pao, L. I., Lam, K. P., Henderson, J. M., Kutok, J. L., Alimzhanov, M., Nitschke, L., Thomas, M. L., Neel, B. G., and Rajewsky, K. (2007). B cell-specific deletion ofprotein-tyrosine phosphatase Shp1 promotes B-1a cell development and causes systemic autoimmunity. Immunity 27, 35-48.

Parravicini, V., Gadina, M., Kovarova, M., Odom, S., Gonzalez-Espinosa, C., Furumoto, Y., Saitoh, S., Samelson, L. E., O'Shea, J. J., and Rivera, J. (2002). Fyn kinase initiates complementary signals required for IgE-dependent mast cell degranulation. Nat Immunol 3, 741-748.

Pasquier, B., Launay, P., Kanamaru, Y., Moura, I. C., Pfirsch, S., Ruffle, C., Henin, D., Benhamou, M., Pretolani, M., Blank, U., et al. (2005). Identification of FcalphaRI as an inhibitory receptor that controls inflammation: dual role of FcRgamma ITAM. Immunity 22, 31-42.

Pinheiro da Silva, F., Aloulou, M., Skurnik, D., Benhamou, M., Andremont, A., Velasco, I. T., Chiamolera, M., Verbeek, J. S., Launay, P., and Monteiro, R. C. (2007). CD16 promotes Escherichia coli sepsis through an FcR gamma inhibitory pathway that prevents phagocytosis and facilitates inflammation. Nat Med 13, 1368-1374.

Reth, M. (1989). Antigen receptor tail clue. Nature 338, 383-384.

Shang, L., Daubeuf, B., Triantafilou, M., Olden, R., Depis, F., Raby, A. C., Herren, S., Dos Santos, A., Malinge, P., Dunn-Siegrist, I., et al. (2014). Selective antibody intervention of Toll-like receptor 4 activation through Fc gamma receptor tethering. J Biol Chem 289, 15309-15318.

Stefanova, I., Hemmer, B., Vergelli, M., Martin, R., Biddison, W. E., and Germain, R. N. (2003). TCR ligand discrimination is enforced by competing ERK positive and SHP-1 negative feedback pathways. Nat Immunol 4, 248-254.

Sugie, K., Jeon, M. S., and Grey, H. M. (2004). Activation of naive CD4 T cells by anti-CD3 reveals an important role for Fyn in Lck-mediated signaling. Proc Natl Acad Sci U S A 101, 14859-14864.

Tan, E. M., Cohen, A. S., Fries, J. F., Masi, A. T., McShane, D. J., Rothfield, N. F., Schaller, J. G., Talal, N., and Winchester, R. J. (1982). The 1982 revised criteria for the classification of systemic lupus erythematosus. Arthritis Rheum 25, 1271-1277.

Tan Sardjono, C., Mottram, P. L., van de Velde, N. C., Powell, M. S., Power, D., Slocombe, R. F., Wicks, I. P., Campbell, I. K., McKenzie, S. E., Brooks, M., et al. (2005). Development of spontaneous multisystem autoimmune disease and hypersensitivity to antibody-induced inflammation in Fcgamma receptor IIa-transgenic mice. Arthritis Rheum 52, 3220-3229.

Utting, O., Teh, S. J., and Teh, H. S. (1998). T cells expressing receptors of different affinity for antigen ligands reveal a unique role for p59fyn in T cell development and optimal stimulation of T cells by antigen. J Immunol 160, 5410-5419.

1. (canceled)

2. The method of claim 14, wherein the leukocytes are granulocytes.

3. The method of claim 14, which comprises detection of the localization of Lyn, Lek, and Fyn, when the presence of Fyn is detected at the membrane it is concluded that the immunoreceptors are in an activating steady state of ITAM signalling.

4. The method of claim 14 which comprises detecting the presence or absence of pSHP-1^{Y537}, pSHP-1^{S591}, pFyn^{Y528}, pFyn^{Y417}, pLyn/Lck^{Y396/397} or pLyn/Lck^{Y508}.

5. The method of claim 4 wherein the presence of pFyn^{Y417} and pSHP-1^{S591} indicates that the immunoreceptors are in an activating steady state of ITAM signalling.

6. The method of claim 4 wherein the presence of pPyn^{Y417} and pSHP-1^{S591} and the absence of pFyn^{Y528} and pSHP-1^{Y436} indicates that the imrnunoreceptors are in an activating steady state of ITAM signalling.

7. The method of claim 4 wherein the presence of nLyn/Lek^{Y396/397} arid pSHP-1^{Y536} indicates that the immunoreceptors are in a steady state of inhibition of ITAMi signaling.

8. The method of claim 4 wherein the presence of pLyn/Lck^{Y396/397} and pSHP-1^{Y536} and the absence of pLyn/Lck^{Y508} and pSHP-1^{S591} indicate that the immunoreceptors are in a steady state of inhibition of ITAMi signaling.

9. The method of claim 14 which comprises detecting the presence or absence of at least one marker selected from the group consisting of pSHP-1^{Y536}, pFyn^{Y528}, pFyn^{Y417}, pSHP-1^{S591} and pPKCα^{Thr638} wherein:
the presence of pSHP-1^{S591} and pPKCα^{Thr638} indicates that the irnmunoreceptors are in an activating steady state of ITAM signalling, or
the presence of pSHP-1^{Y536} indicates that the immunoreceptors are in an inhibiting a steady state of inhibition of ITAM signalling, or
the absence of pFyn^{Y528} and the presence pFyn^{Y417} indicates that the immunoreceptors are in an activating steady state of ITAM signalling, or
the presence of Lyn/Lck^{Y396/397} indicates that the irnmunoreceptors are in an inhibiting a steady state of inhibition of ITAM signalling, or
the presence of Lyn/Lck^{Y396/397} and the absence of Lyn/Lck^{Y508} indicates that that the immunoreceptors are in a steady state of inhibition of ITAM signalling.

10. The method of claim 14 which combines detection of the localization of Fyn and Lyn/Lck and the detection of at least one marker selected from the group consisting of pSHP-1^{Y536}, pFyn^{Y528}, pFyn^{Y417}, pSHP-1^{S591} and pPKCα^{Thr638}.

11. The method of claim 14 wherein the detection is determined by a flow cytometric and/or image stream method.

12. The method of claim 14 which further comprises detecting the presence of absence of at least one phenotypic marker of B-Cell, T-cell, monocytes, neutrophils and basophils.

13. The method of claim 14 which further comprises detecting the presence or absence of at least one intracellular cytokinelchemokine.

14. A method for the diagnosis and treatment of an inflammatory autoimmune disease in a patient comprising
i) determining whether the immunoreceptors in a population of leukocytes in a blood sample obtained from the patient are associated with an activated or inactivated form of the kinases Fyn and Lyn/Lck and an activated or inactivated form the phosphatase SHP-1: and
ii) concluding that administering a treatment to a patient the whose immunoreceptors are associated with an active form of Fyn and an inactivated form of SHP-1 and thus are in a steady state of activation of ITAM signaling (ITAMa).

15. A method for determining whether a patient suffering from an inflammatory autoimmune disease achieves a response with a treatment which comprises
i) diagnosing and treating the patient according to the method of claim 14,
ii) providing a blood sample of the patient after a period of treatment, and
iii) concluding that the patient has achieved a response to the treatment when the immunoreceotors of the leukocytes present in the blood sample have returned to a steady state of inhibition of ITAM signaling or concluding that the patient has not achieved a response to the treatment when the immunoreceptors of the leukocytes present in the blood sample are maintained in the steady state of activation of ITAM signaling.

16. The method of claim 2, wherein the granulocytes are neutrophils, eosinophils, basophils, mononuclear phagocytes, or lymphocytes.

17. The method of claim 16, wherein the lymphocytes are B cells, T cells or natural killer (NK) cells.

18. A method for analysing steady-state activation or inhibition of immunoreceptor tyrosine-based activation motif (ITAM) signalling of immunoreceptors in leukocytes, comprising
detecting activated or inactivated forms as of the kinases Fyn and Lyn/Lck in the leukocytes,
wherein the leukocytes are obtained from a patient who has or is suspected of having an inflammatory autoimmune disease,
and wherein detection of an active form of Fyn indicates that the ITAM signalling of immunoreceptors is in a steady state of activation, and detection of an active form of kinase Lyn/Lck indicates that the ITAM signalling of immunoreceptors is in a steady state of inhibition.
